(19) United States
(12) Patent Application Publication
(10) Pub. No.: US 2013/0231528 A1
(43) Pub. Date: Sep. 5, 2013

(54) PROTECTIVE SLEEVE AND ASSOCIATED SURGICAL METHOD

(71) Applicant: DAN VOIC, Cedar Grove, NJ (US)

(72) Inventor: DAN VOIC, Cedar Grove, NJ (US)

(73) Assignee: MISONIX INCORPORATED, Farmingdale, NY (US)

(21) Appl. No.: 13/411,839

(22) Filed: Mar. 5, 2012

(51) Int. Cl. A61B 17/22; A61B 1/06

(52) U.S. Cl. 600/104; 606/128

(57) ABSTRACT

An ultrasonic surgical instrument includes an elongate substantially rigid probe and an elongate tubular sheath member. The probe is operatively connected at a proximal end to a source of ultrasonic mechanical vibrations and has a distal tip configured for transmitting ultrasonic vibration energy into organic tissues. The probe longitudinally traverses the sheath member. The sheath member has a distal edge with a first portion on one side of the probe and a second portion on an opposite side of the probe. The first portion of the distal edge of the sheath member is disposed substantially closer than the second portion of the sheath's distal edge to the distal tip of the probe. The second portion of the distal edge is so spaced from the distal tip as to permit effective visualization of the distal tip during use of the instrument.

### BACKGROUND OF THE INVENTION

This invention relates to ultrasonic surgical instruments and associated surgical methods. More particularly, this invention relates to a protective sleeve and an associated medical treatment probe. These probes may be ultrasonic vibratory tools that fragment and emulsify hard and soft tissue in a clinical environment, the protective sleeves reducing collateral tissue damage and unwanted heat propagation.

Over the past 30 years, several ultrasonic tools have been invented which can be used to ablate or cut tissue in surgery. Such devices are disclosed by Wuchinich et al. in U.S. Pat. No. 4,223,676 and Idemoto et al in U.S. Pat. No. 5,188,102.

In practice, these surgical devices include a blunt tip hollow probe that vibrates at frequencies between 20 kc and 100 kc, with amplitudes up to 300 microns or more. Such devices ablate tissue by producing cavitation bubbles which implode and disrupt cells, by generating tissue compression and relaxation stresses (sometimes called the jackhammer effect), or by giving rise to other forces such as mechanical shearing and micro streaming of bubbles in the tissue matrix. The effect is that the tissue becomes fragmented and separated. It then becomes emulsified with the irrigant solution. The resulting emulsion is then aspirated from the site. Bulk excision of tissue is possible by applying the energy around and under an unwanted tissue mass to separate it from the surrounding structure. The surgeon can then lift the tissue out using common tools such as forceps.

The probe or tube is excited by a transducer of either the piezoelectric or magnetostrictive type that transforms an alternating electrical signal within the frequencies indicated into a longitudinal or transverse vibration. When the probe is attached to the transducer, the two become a single element with series and parallel resonances. The designer will try to tailor the mechanical and electrical characteristics of these elements to provide the proper frequency of operation. Most of the time, the elements will have a long axis that is straight and has the tip truncated in a plane perpendicular to the long axis, as shown in FIG. 1. This is done for simplicity and economic considerations. In almost all applications, whether medical or industrial, such an embodiment is practical and useful. However, in applications such as the debridement of burns, wounds, diabetic ulcers or ulcers induced by radiation treatments, the blunt straight probe has been shown to be less effective in removing the hard eschar buildup that occurs when the wound is healing. This eschar buildup must be removed so that the healthy tissue is exposed and allowed to close the wound to provide complete healing with minimal scar tissue formation. Also, the small diameter tip, since it is cannulated, has a small annular area with limits energy transmission into the wound.

Almost all ultrasonic probes used for tissue removal are completely covered by a protective sleeve except for a small segment at the distal end of the probes. The exposed area is needed for the effective engagement, penetration and removal of the target tissue. The larger or longer the exposed segment, the easier for the surgeon to see the instrument's end and to operate on the target tissue and only the target tissue. However, large exposed areas greatly increase the risk of unwanted probe-tissue contact, away from the intended target.

### OBJECTS OF THE INVENTION

It is an object of the present invention to provide an improved ultrasonic probe assembly.

It is a more particular object of the present invention to provide an ultrasonic probe assembly that enables adequate visualization of the instrument's distal end and yet provides protection against undesired probe-tissue contact outside of the target area.

These and other objects of the invention will be apparent from the drawings and descriptions herein. Although every object of the invention is attained in at least one embodiment of the invention, there is not necessarily any embodiment which attains all of the objects of the invention.

### SUMMARY OF THE INVENTION

An ultrasonic surgical instrument in accordance with the present invention comprises an elongate substantially rigid probe and an elongate tubular sheath member. The probe is operatively connected at a proximal end to a source of ultrasonic mechanical vibrations and has a distal tip configured for transmitting ultrasonic vibration energy into organic tissues. The probe longitudinally traverses the sheath member. The sheath member has a distal edge with a first portion on one side of the probe and a second portion on an opposite side of the probe. The first portion of the distal edge of the sheath member is disposed substantially closer than the second portion of the sheath's distal edge to the distal tip of the probe. The second portion of the distal edge is so spaced from the distal tip as to permit effective visualization of the distal tip during use of the instrument.

Typically, the first portion of the distal edge of the sheath member is spaced between 1 mm and about 3 mm from the distal tip of the probe, while the second portion of the distal edge of the sheath member is spaced between about 8 mm and 12 mm from the distal tip of the probe.

Where the first portion of the distal edge of the sheath member subtends a first angle about a longitudinal axis of the probe, and the second portion of the distal edge of the sheath member subtends a second angle about the longitudinal axis of the probe, the first angle is between about 160° and about 270°, while the second angle is between about 90° and about 200°. Generally, the first portion of the sheath's distal edge has an angular extent sufficient to insulate organic tissues of a patient from undesirable contact with the probe, while the second portion of the sheath's distal edge has a sufficient angular extent to permit visualization of the distal tip of the probe and its proximity to tissue of the patient.

Concomitantly, the present invention is also directed to a protective sleeve for use with an elongate substantially rigid probe where the probe is operatively connected at a proximal end to a source of ultrasonic mechanical vibrations and has a distal tip configured for transmitting ultrasonic vibration energy into organic tissues. The protective sleeve comprises an elongate tubular sheath member having a distal edge with a first portion on one side and a second portion on an opposite side. The first portion of the sheath's distal edge is disposed substantially distally of the second portion. The sheath member is so dimensioned that the probe when longitudinally inserted into the sheath member has its distal tip extending distally beyond the first portion (the distal-most portion) of the distal edge of the sheath member. The second portion of the distal edge of the sheath member is so spaced from the first portion along a longitudinal axis of the sheath member as to permit effective visualization of the distal tip of the probe on the opposite side of the sheath member.

The sheath member is further so dimensioned that, during use of the probe with the sleeve, the probe protrudes a distance of between about 1 mm and about 3 mm beyond the first portion of the distal edge of the sheath member, while the second portion of the distal edge of the sheath member is spaced between about 8 mm and 12 mm from the distal tip of the probe. As discussed above, the first portion of the distal edge of the sheath member preferably subtends an angle between about 160° and about 270° about the longitudinal axis of the sheath member, while the second portion of the distal edge of the sheath member subtends an angle between about 90° and 200° about the longitudinal axis.

A surgical method in accordance with the present invention comprises (i) inserting an endoscope into a patient, and (ii) inserting an ultrasonic probe into the patient, the probe having a distal tip and being substantially surrounded by a sheath, the sheath having a distal edge with a recess defining a distal edge portion that is spaced from the distal tip of the probe to allow visualization of the distal end thereof. The method further comprises (iii) orienting the sheath about the probe and relative to the endoscope so that the distal tip of the probe is visible through the recess via the endoscope.

A surgical method in accordance with the present invention more particularly comprises (a) providing an elongate substantially rigid probe operatively connected at a proximal end to a source of ultrasonic mechanical vibrations, the probe having a distal tip configured for transmitting ultrasonic vibration energy into organic tissues, and (b) providing the probe with an elongate tubular sheath member, wherein the probe longitudinally traverses the sheath member, the sheath member having a distal edge with a first portion on one side of the probe and a second portion on an opposite side of the probe, the first portion of the distal edge being disposed substantially closer than the second portion of the distal edge to the distal tip of the probe, the second portion of the distal edge being so spaced from the distal tip as to permit effective visualization of the distal tip during use of the instrument. Then the surgical method also particularly comprises (c) inserting a distal end portion of the probe together with the sheath into a patient, (d) inserting a distal end portion of an endoscope into the patient, (e) positioning the probe, the sheath and the endoscope so that the endoscope is located on the same side of the probe as the second portion (recessed or shorter portion) of the distal edge of the sheath, (f) utilizing the endoscope to visualize the distal tip of the probe distally of the second portion of the distal edge of the sheath, and (f) during the utilizing the endoscope to visualize the distal tip of the probe, bringing the distal tip into contact with organic tissue of the patient and energizing the probe with ultrasonic vibration energy to effectuate a surgical operation on the organic tissue under visualization via the endoscope.

A protective sleeve in accordance with the present invention maintains the probe exposure needed for effective tissue removal, improves the visibility of the probe's unprotected area and prevents the occurrence of unwanted probe-tissue contact.

### BRIEF DESCRIPTION OF THE DRAWING

FIG. 1 is partially a schematic perspective view and partially a block diagram of an ultrasonic surgical instrument assembly in accordance with the present invention.

FIG. 2 is a distal end elevational view of an ultrasonic probe and sheath shown in FIG. 1.

FIG. 3 is a schematic side elevational view, on a smaller scale, of the ultrasonic probe and sheath of FIGS. 1 and 2, showing a preferred disposition thereof relative to an endoscope during a surgical procedure.

### DETAILED DESCRIPTION

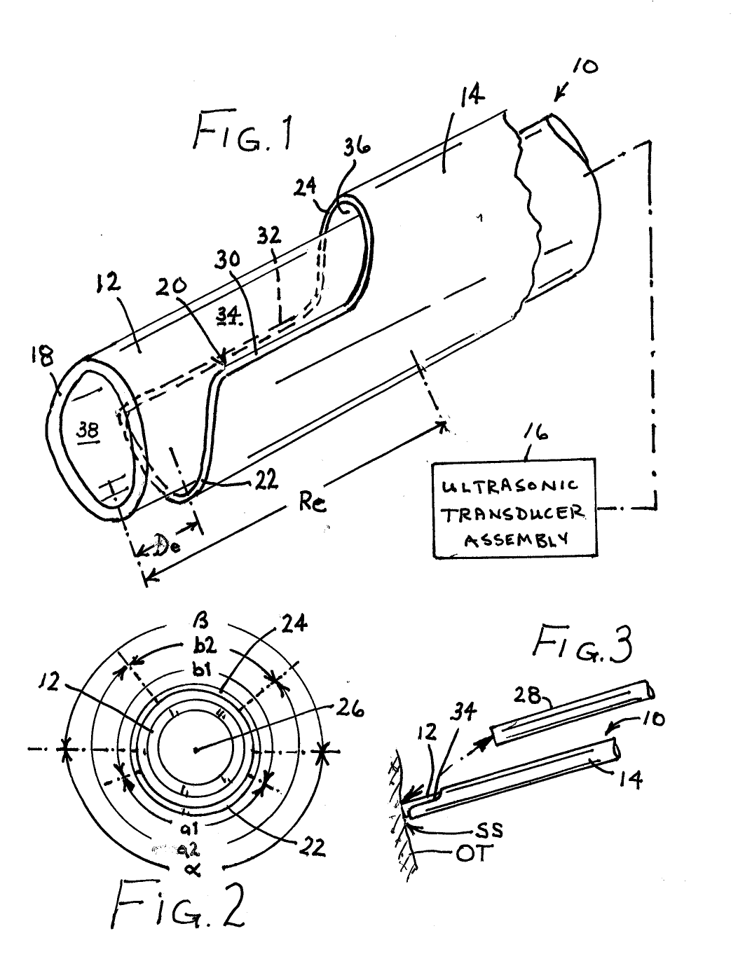

As depicted in FIG. 1, an ultrasonic surgical instrument assembly 10 comprises an elongate substantially rigid probe 12 and an elongate tubular sheath member 14. Probe 12 is operatively connected at a proximal end to a source 16 of ultrasonic mechanical vibrations and has a distal tip 18 configured for transmitting ultrasonic vibration energy into organic tissues OT (FIG. 3). Probe 12 longitudinally traverses sheath member 14.

Sheath member 14 has a distal edge 20 with a first portion 22 on one side of probe 12 and a second portion 24 on an opposite side of the probe. Edge portion 22 is located distally of edge portion 24 and is therefore disposed substantially closer than edge portion 24 to distal tip 18 of probe 12. Sheath edge portion 24 is so spaced from probe distal tip 18 as to permit effective visualization of the distal tip during use of the instrument assembly 10.

Edge portion 22 of sheath distal edge 20 is typically spaced a distance De of between 1 mm and about 3 mm from distal tip 18 of probe 12. Sheath distal edge portion 24 is typically spaced a distance Re of between about 8 mm and 12 mm from probe tip 18.

As depicted in FIG. 2, edge portion 22 of sheath distal edge 20 may subtend an angle α about a longitudinal axis 26 of probe 12 that is between an angle a1 of about 160° and an angle a2 of about 270°. Concomitantly, edge portion 22 of sheath distal edge 20 may subtend an angle β about longitudinal axis 26 that is between an angle b1 of about 200° and an angle b2 of about 90°. Edge portion 22 of sheath distal edge 20 preferably has an angular extent α sufficient to insulate organic tissues OT of a patient from undesirable contact with probe 12, while edge portion 24 has a sufficient angular extent β to permit visualization of distal tip 18 and its proximity to tissues OT of the patient. In FIG. 1, subtended angles α and β are shown at values of about 180° each.

Sheath member 14 constitutes a protective sleeve wherein distal edge 20 has longitudinally staggered edge portions 22 and 24. Thus, sheath member is longer on the side of relatively distal edge portion 22 than on the side of relatively proximal edge portion 24. Proximal edge portions 22 and 24 each take the approximate form of a circular section or arc. Edge portions 22 and 24 are connected to one another by longitudinally extending linear edge sections 30 and 32 (FIG. 1). Proximal edge portion 24 defines, together with linear edge sections 30 and 32, a cutout, recess or window 34 that enable and facilitates viewing of the probe's distal tip 18 during a surgical procedure (FIG. 3).

Sheath member 14 is so dimensioned that probe 12, when longitudinally inserted into the sheath member, has its distal tip 18 extending distally the distance De beyond sheath edge portion 22. Edge portion 24 of sheath distal edge 20 is so spaced from distal probe tip 18 by the distance Re as to permit effective visualization of the distal tip via cutout, recess or window 34.

Sheath member 14 constitutes a protective sleeve for use particularly with an elongate substantially rigid probe 12 in a laparoscopic or endoscopic procedure under indirect visualization via an endoscope 28 (FIG. 3). In a surgical method utilizing sheath or sleeve 14, one inserts endoscope 28 into a patient, and also insert ultrasonic probe 12 with sheath 14. One arranges or orients sheath 14 about probe 12 and relative to endoscope 28 so that distal tip 18 of the probe is visible through cutout, recess or window 34 via endoscope 28. In other words, one positions probe 12, sheath 14 and endoscope 28 so that the endoscope is located on the same side of the probe as sheath edge portion 24. During the surgical operation, endoscope 28 is utilized to visualize distal tip 18 of probe 12 through cutout, recess or window 34, transducer assembly 16 is operated to generate a standing ultrasonic vibratory wave in probe 12, and probe 12 is manipulated to place distal tip 18 into contact with target organic tissues OT at surgical site SS.

Probe 12 may take the form of a cannula, as illustrated in FIG. 1. A coolant such as saline solution may be guided to the surgical site SS via a lumen 36 between an outer surface of probe 12 and an inner surface of sheath 14. Suction may be applied to a lumen 38 of probe 12, from a proximal end thereof, to remove tissue fragments, spent coolant and other debris from the surgical site SS.

Although the invention has been described in terms of particular embodiments and applications, one of ordinary skill in the art, in light of this teaching, can generate additional embodiments and modifications without departing from the spirit of or exceeding the scope of the claimed invention. It is to be noted, for instance, that the principles of the present invention may be applied to surgical instruments other than ultrasonic probes, such as laparoscopic cauterization tools, which may include a sleeve or sheath extending to the distal end of the device with an operative tip protruding from the distal end of the sleeve or sheath during use of the instruments. In addition, surgical instruments in accordance with the invention may be used in open surgery such as wound debridement rather than exclusively in endoscopic operations.

Accordingly, it is to be understood that the drawings and descriptions herein are proffered by way of example to facilitate comprehension of the invention and should not be construed to limit the scope thereof.

What is claimed is:

1. An ultrasonic surgical instrument comprising:
an elongate substantially'rigid probe operatively connected at a proximal end to a source of ultrasonic mechanical vibrations, said probe having a distal tip configured for transmitting ultrasonic vibration energy into organic tissues; and
an elongate tubular sheath member, said probe longitudinally traversing said sheath member, said sheath member having a distal edge with a first portion on one side of said probe and a second portion on an opposite side of said probe, said first portion of said distal edge being disposed substantially closer than said second portion of said distal edge to said distal tip of said probe, said second portion of said distal edge being so spaced from said distal tip as to permit effective visualization of said distal tip during use of said instrument.

2. The instrument defined in claim 1 wherein said first portion of said distal edge of said sheath member is spaced between 1 mm and about 3 mm from said distal tip of said probe, said second portion of said distal edge of said sheath member being spaced between about 8 mm and 12 mm from said distal tip of said probe.

3. The instrument defined in claim 1 wherein said first portion of said distal edge of said sheath member subtends a first angle about a longitudinal axis of said probe, said second portion of said distal edge of said sheath member subtending a second angle about said longitudinal axis of said probe, said first angle being between about 160° and about 270°, said second angle being between about 90° and about 200°.

4. A protective sleeve for use with an elongate substantially rigid probe operatively connected at a proximal end to a source of ultrasonic mechanical vibrations and having a distal tip configured for transmitting ultrasonic vibration energy into organic tissues, comprising an elongate tubular sheath member having a distal edge with a first portion on one side and a second portion on an opposite side, said first portion being disposed substantially distally of said second portion, said sheath member being dimensioned so that said probe is longitudinally insertable in said sheath member with the distal tip of said probe extending distally beyond said first portion of said distal edge of said sheath member, said second portion of said distal edge of said sheath member being so spaced from said first portion along a longitudinal axis of said sheath member as to permit effective visualization of said distal tip of said probe on said opposite side of said sheath member.

5. The sleeve defined in claim 4 wherein said sheath member is so dimensioned that, during use of said probe with the sleeve, said probe protrudes a distance of between about 1 mm and about 3 mm beyond said first portion of said distal edge of said sheath member, while said second portion of said distal edge of said sheath member is spaced between about 8 mm and 12 mm from said distal tip of said probe.

6. The sleeve defined in claim 4 wherein said first portion of said distal edge of said sheath member subtends a first angle about said longitudinal axis of said sheath member, said second portion of said distal edge of said sheath member subtending a second angle about said longitudinal axis, said first angle being between about 160° and about 270°, said second angle being between about 90° and 200°.

7. A surgical method comprising:
providing an elongate substantially rigid probe operatively connected at a proximal end to a source of ultrasonic mechanical vibrations, said probe having a distal tip configured for transmitting ultrasonic vibration energy into organic tissues;
providing said probe with an elongate tubular sheath member, wherein said probe longitudinally traverses said sheath member, said sheath member having a distal edge with a first portion on one side of said probe and a second portion on an opposite side of said probe, said first portion of said distal edge being disposed substantially closer than said second portion of said distal edge to said distal tip of said probe, said second portion of said distal edge being so spaced from said distal tip as to permit effective visualization of said distal tip during use of said instrument;
inserting a distal end portion of said probe with said sheath into a patient;
inserting a distal end portion of an endoscope into the patient;
positioning said probe, said sheath and said endoscope so that said endoscope is located on the same side of said probe as said second portion of said distal edge;
utilizing said endoscope to visualize said distal tip distally of said second portion of said distal edge of said sheath;
during the utilizing said endoscope to visualize said distal tip, bringing said distal tip into contact with organic tissue of the patient and energizing said probe with ultrasonic vibration energy to effectuate a surgical operation on said organic tissue under visualization via said endoscope.

8. A surgical method comprising:
inserting an endoscope into a patient;
inserting an ultrasonic probe into the patient, said probe having a distal tip and being surrounded by a sheath, said sheath having a distal edge with a recess defining a distal edge portion that is spaced from said distal tip of said probe to allow visualization of said distal end; and
orienting said sheath about said probe and relative to said endoscope so that said distal tip of said probe is visible through said recess via said endoscope.
